(19) United States
(12) Patent Application Publication
(10) Pub. No.: US 2003/0111507 A1
(43) Pub. Date: Jun. 19, 2003

(54) Balloon actuator for use in a resectioning device

(76) Inventor: George Nunez, Miami, FL (US)

Correspondence Address: Fay Kaplun & Marcin, LLP, 17th Floor, 100 Maiden Lane, New York, NY, 10038, US

(21) Appl. No.: 10/017,197

(22) Filed: Dec. 14, 2001

(51) Int. Cl. A61B 17/00

(52) U.S. Cl. 227/180.1

(57) ABSTRACT

An actuator for a resection device comprises a housing which, when in an operative position, is located within a body lumen of a patient and an inflatable element having a collapsed configuration and an expanded configuration, wherein a characteristic length of the inflatable element is greater when in the expanded configuration than when in the collapsed configuration, a first surface of the inflatable element contacting the housing and a second surface of the inflatable element contacting a moveable component of the device movably coupled to the housing such that changing the configuration of the inflatable element between the expanded and collapsed configurations moves the moveable component relative to the housing in combination with a fluid supply line extending from a distal end connected to the inflatable element to a proximal end which, when the housing is in the operative position, remains outside the body of the patient.

### FIELD OF THE INVENTION

The present invention relates to resection devices for performing localized resections of lesions in tubular organs. In particular, the invention relates to actuators for the various components for such devices.

### DESCRIPTION OF RELATED ART

A resection procedure involves excising a portion of an organ and removing the excised tissue. In procedures under which a full thickness section of an organ is removed (i.e., a full-thickness resection procedure), in order to maintain the interior of the organ sealed, tissue surrounding the resected portion must also be bonded together to close the hole created by the resection. Various conventional devices and procedures are available for resectioning lesions in tubular organs.

For example, several known resection devices and procedures require at least one incision in an area near the portion of the organ to be excised to provide access to the lesion or treatment site (because, for example, the resectioning device may lack sufficient steering and/or flexibility). Thus, such an incision may be required to allow the physician to access the organ section to be excised and guide the device to that section.

Other resectioning devices includes stapling and cutting assemblies mounted on a shaft which may be bent or formed into a desired shape and then inserted into a patient's body cavity. The shafts of such devices are formed so that, once the shaft has been bent into the desired shape, the rigidity of the shaft ensures that shape is maintained throughout the operation. This arrangement limits the effective operating range of the device as the bending of the shaft into the desired shape before insertion and the rigidity of the shaft once bent require the physician to ascertain the location of the organ section to be removed before insertion, and to deform the shaft accordingly.

Furthermore, the rigidity of the shaft makes it difficult to reach remote areas—particularly those areas which must be reached by winding and/or circuitous routes (e.g., sigmoid colon). Thus, one or more incisions may be required near the organ section to be excised in order to properly position the device.

### SUMMARY OF THE INVENTION

The present invention is directed to an actuator for a resection device comprising a housing which, when in an operative position, is located within a body lumen of a patient and an inflatable element having a collapsed configuration and an expanded configuration, wherein a characteristic length of the inflatable element is greater when in the expanded configuration than when in the collapsed configuration, a first surface of the inflatable element contacting the housing and a second surface of the inflatable element contacting a moveable component of the device movably coupled to the housing such that changing the configuration of the inflatable element between the expanded and collapsed configurations moves the moveable component relative to the housing in combination with a fluid supply line extending from a distal end connected to the inflatable element to a proximal end which, when the housing is in the operative position, remains outside the body of the patient.

### BRIEF DESCRIPTION OF THE DRAWINGS

The accompanying drawings are included to provide a further understanding of the invention and are incorporated in and constitute part of the specification, illustrate several embodiments of the invention and, together with the description, serve to explain examples of the present invention. In the drawings:

FIG. 1 is a schematic side view of an anvil actuator for an FTRD, in the open position;

FIG. 2 is a schematic side view of the anvil actuator for an FTRD as shown in FIG. 1, in the closed position;

FIG. 3 is a schematic side view of a staple firing mechanism actuator for an FTRD;

FIG. 4 is a schematic side view of a cutting blade actuator for an FTRD; and

FIG. 5 is a schematic side view of another embodiment of a cutting blade actuator for an FTRD.

### DETAILED DESCRIPTION OF EMBODIMENTS OF THE INVENTION

The present invention is an actuator for use in a resectioning device (e.g. a full-thickness resectioning device “FTRD”) that uses force generated by an expandable component to operate one or more components of the device. Those skilled in the art will understand that the actuator according to the present invention may be used in conjunction with any resectioning device and that the following examples which describe the use of the actuator(s) in an FTRD are merely illustrative of one possible use. For example, the actuator may be used to open and close an operating head of a resectioning device, by changing a distance between an anvil portion and a stapler housing thereof. Alternatively or additionally, the actuator nay operate a staple firing mechanism and/or the cutting blade of the device.

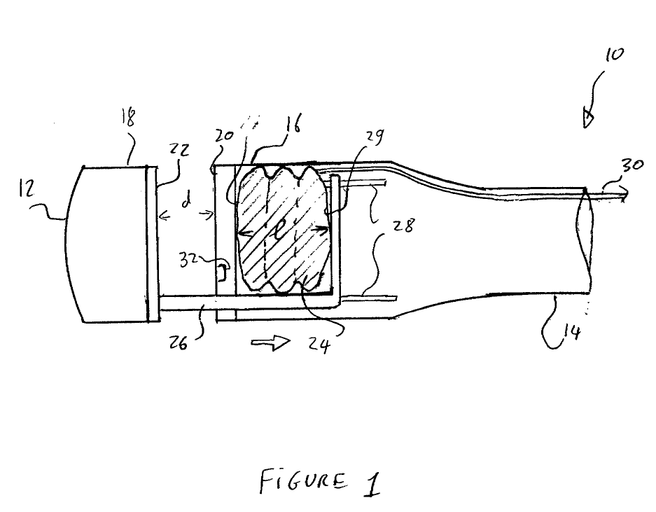

With reference to FIG. 1, an FTRD 10 shown in this example includes an anvil portion 18 and a stapler housing 16. The anvil portion 18 defines a distal end 12 of the FTRD 10 which is introduced within a body lumen of a patient either via a natural body orifice or through a incision. A flexible portion 14 of the FTRD 10 extends proximally from the stapler housing 16 to a proximal end which remains outside the patient's body during the procedure. One or more working channels extend through the flexible portion 14 and the stapler housing 16, and may extend from the proximal end to the distal end 12 of the FTRD 10.

The stapler housing 16 includes a plurality of staple slits 20 formed therein to allow staples 32 to exit the stapler housing 16 and enter tissue received adjacent thereto. Staple forming grooves 22 are formed on a surface of the anvil portion 18 that faces the staple slits 20, and are formed so that, when legs of each of the staples 32 are driven against a respective staple forming groove 22, they are bent into a desired shape to couple portions of tissue through which they have passed to one another.

During insertion of the FTRD 10 into a body lumen, a gap “d” between the anvil portion 18 and the stapler housing 16 is maintained at a minimum closed position to facilitate the insertion. In this closed position, the gap “d” is preferably reduced until the stapler housing 16 contacts the anvil portion 18. Once the device has reached a desired location within the body lumen, the anvil portion 18 is moved relative to the stapler housing 16 to increase the size of the gap “d” until the anvil portion 18 and the stapler housing 16 are in an open, tissue-receiving position with respect to one another.

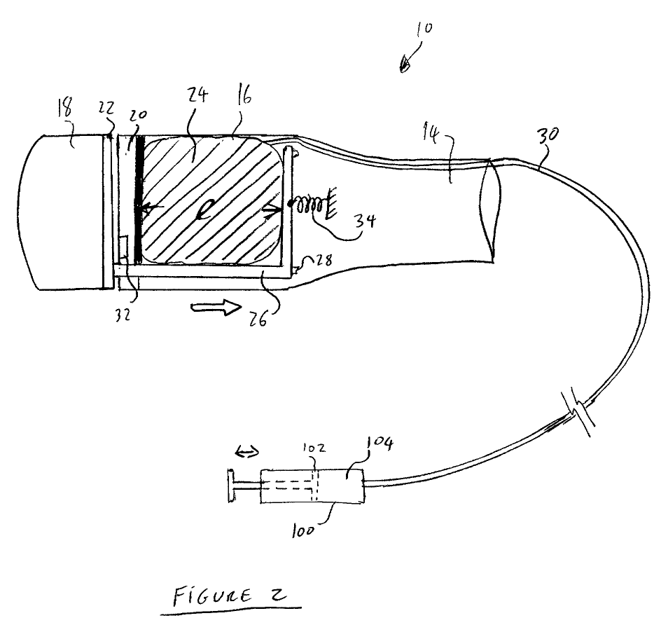

When the anvil portion 18 and the stapler housing 16 have reached the tissue-receiving position, a portion of tissue to be resected may be drawn by an operator through the gap “d” using known methods into a tissue-receiving space formed within the FTRD 10. FIG. 1 shows the FTRD 10 with the anvil portion 18 and the stapler housing 16 in the tissue-receiving configuration. The anvil portion 18 and the stapler housing 16 are then moved toward one another until the gap “d” reaches a desired stapling gap size wherein tissue adjacent to the tissue to be resected is gripped in the gap “d”. At this point tissue stapling and resecting procedures may be performed. Once these procedures have been completed, the size of the gap “d” may be slightly increased to release the clamped tissue and then closed to maintain the resected tissue in the tissue receiving space of the FTRD 10 during withdrawal of the FTRD 10 from the body lumen. The actuator mechanisms to accomplish these relative movements of the anvil portion 18 and the stapler housing 16 are described in more detail below. As will be understood by those of skill in the art, the size of the gap “d” in the stapling position may be controlled by configuring the anvil portion 18 and the stapler housing 16 so that they cannot be drawn together past the distance “d”. That is, the fully closed position shown in FIG. 2 may also be the stapling position with the gap “d” representing the minimum separation between the anvil portion 18 and the stapler housing 16.

In one exemplary embodiment, an expandable component is used as an actuator to move the anvil portion 18 relative to the stapler housing 16 to open and close the gap. Those skilled in the art will understand that the actuator according to the present invention may just as easily move the stapler housing 16 relative to the anvil portion 18 to achieve the same results, or may move both the anvil portion 18 and the stapler housing 16 relative to one another.

The expandable component may, for example, include an inflatable balloon 24 having a first face 27 contacting an abutting surface of the stapler housing 16 and a second face 29 contacting an abutting surface of a bracket 26 connected to the anvil portion 18. The bracket 26 is moveably coupled to the stapler housing 16 (and the rest of the FTRD 10) so that, when the expandable component is deployed from a collapsed configuration to an expanded configuration, the bracket 26 and the anvil portion 18 are moved relative to the rest of the stapler housing 16 and the rest of the FTRD 10.

Specifically, when the balloon 24 is inflated from the collapsed configuration to the expanded configuration, its characteristic length “I” increases. The characteristic length may be defined as a length of the balloon 24 along an axis of maximum expansion. For example, the characteristic length of a spherical balloon which expands equally in all directions would be the diameter, while the characteristic length “I” of an ellipsoidal balloon may, for example, be a length of the major axis thereof.

As the balloon 24 is inflated, the bracket 26 is pushed along tracks 28, and the anvil portion 18 is moved toward the staple slits 20. As described above, FIG. 2 shows the FTRD 10 in the closed/stapling configuration with the balloon 24 expanded so that the length “I” is at a maximum. In this position, the gap “d” is reduced to a desired stapling distance as described above. Fluid supply lines 30 are used to carry pressurized hydraulic fluid to balloon 24, and preferably extend along flexible catheter 14 to the outside ambient. Those skilled in the art will understand that check valves and other flow control devices may be included in fluid lines 30, as required, for example, to allow an operator to choose a degree of expansion of the balloon 24 and a corresponding stapling gap size “d” which is greater than the minimum distance shown in FIG. 2.

A spring 34 or another resilient element may be used to bias the anvil portion 18 in a direction opposite from the direction in which the anvil portion 18 is moved as the balloon 24 is inflated. Thus, the force required to increase the size of the gap “d” from the closed or the stapling position to the open position is provided by the spring 34 and the balloon 24 does not have to provide a force to increase the size of gap “d”, but only to reduce it. In a different embodiment, as would be understood by those of skill in the art, an opposite configuration may be used with a balloon configured as with the balloon 24 providing a force to increase the size of the gap “d” with one or more resilient members operating to reduce the size of the gap “d”.

FIG. 2 shows an exemplary embodiment of a source of pressurized fluid which may be used to inflate balloon 24. A hand operated pump or syringe 100 is connected to fluid lines 30, and includes a movable piston 102, which when actuated by an operator, pushes fluid contained in the cavity 104 into the fluid lines 30. As would be understood by those of skill in the art, any suitable configuration of piston or other pump apparatus may be used which is capable of providing sufficient fluid to inflate the balloon 24.

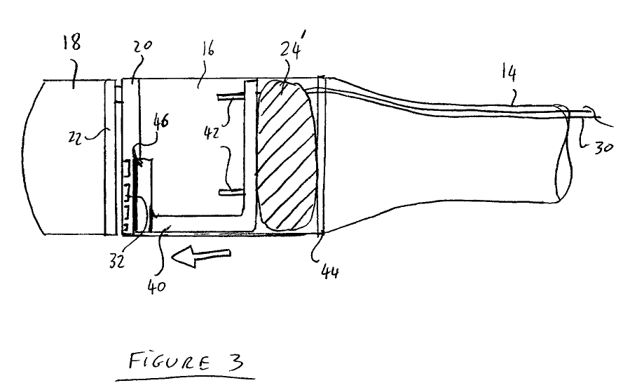

A different embodiment of an FTRD including an actuator according to the present invention is shown in FIG. 3. A balloon 24′ according to this example, when inflated actuates a mechanism for firing the staples 32 from the housing 16 through the staple slits 20 into the tissue to be stapled, and against the staple forming grooves 22 of the anvil portion 18. The balloon 24′ has a first surface abutting a flange 44 fixed relative to the housing 16 and a second surface pressing against a staple actuator 40. As the balloon 24′ is inflated, the staple actuator 40 is pushed along guides 42, in the direction shown by the arrow in FIG. 3, in a telescoping motion relative to the stapler housing 16. The staple actuator 40 in turn has a staple cam surface 46 that presses the staples 32 out of the staple slits 20.

Operation of the actuator shown in FIG. 3, fires all of the staples 32 substantially simultaneously. However, as would be understood by those of skill in the art, the staple cam 46 of the staple actuator 40 may be shaped inflation of the balloon 24′ fires each of the staples sequentially. For example, the cam surface 46 may be angled, or may include protrusions of different lengths so that the individual staples are contacted at different times during the inflation of the balloon 24′.

Furthermore, those of skill in the art will understand that the balloons 24 and 24′ may be formed in any desired shape and may be configured so that expansion of the balloons 24, 24′ is directed substantially along a desired axis. For example, the balloons 24, 24′ may be formed in a toroidal shape and configured to expand principally along the longitudinal axis of the FTRD 10 while minimizing radial expansion.

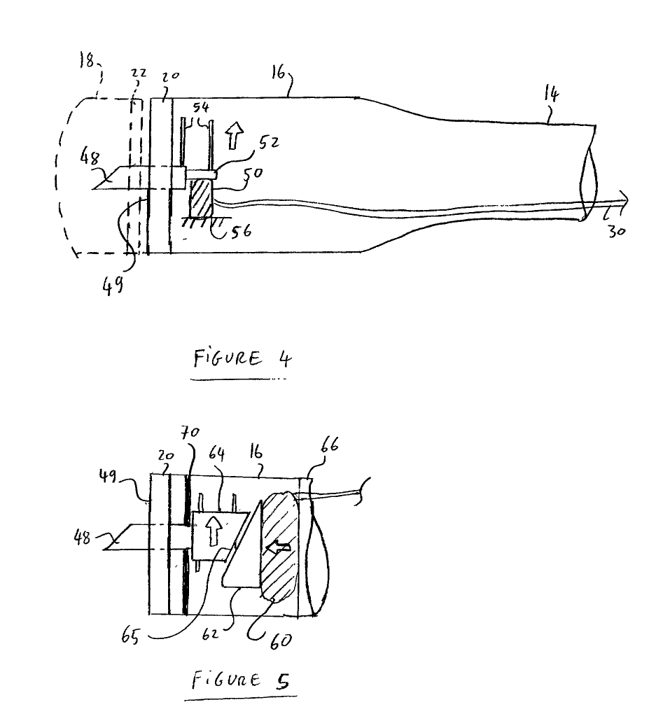

Yet another actuator mechanism according to the invention may be used to actuate a cutting blade of an FTRD. As shown in FIG. 4, a blade 48 is located within the housing 16, protruding from a slot 49. Guides 54 further direct movement of blade 48 along the perimeter of the housing 16 to control the path along which the tissue is cut. In a typical use of the FTRD, the tissue to be excised is first drawn inside the device using known methods, the area surrounding the tissue is stapled, and the tissue is cut, either after or simultaneously with the stapling operation.

In this exemplary embodiment, an inflatable balloon 50 is disposed within the housing 16, abutting a surface 56 fixed relative to the housing 16. When the balloon 50 is inflated, it expands in the direction shown by the arrow in FIG. 4 pressing on an actuating surface 52 that is connected to the blade 48. In this example, the balloon 50 may be designed to expand preferentially substantially circumferentially.

FIG. 5 shows another embodiment of an actuating balloon that deploys a cutting blade of an FTRD. In this example, a balloon 60 expands principally along a longitudinal axis of the FTRD when inflated. This longitudinal expansion is transformed into a circumferential movement of the blade 48 by cam surfaces as described below. A first cam 62 is connected to the balloon 60, and is sloped as shown in the FIG. 4. A second cam 64 is connected to the blade 48, and has a sloped surface 65 that matches the first cam 62. When the balloon 60 is inflated, the action of the first cam 62 on the second cam 64 causes the blade 48 to move circumferentially, in the desired manner, to cut tissue along a desired path.

It may be desirable to completely contain the blade 48 within the housing 16 until the moment it is required for cutting. In this case, a first step of the cutting process may be to deploy the blade 48 from the housing 16 by, for example, using inflatable balloons as described above. For example, guides 54 shown in FIG. 4 may include an angled initial portion that directs the blade 48 longitudinally through the slot 49, towards the anvil portion 18, as the balloon 60 starts to inflate. A similar effect may be obtained by properly shaping the first and second cams 62, 64 as shown in FIG. 5. For example, cam 64 may be constrained to move longitudinally by action of the cam 62 until a barrier 70 is reached, at which point the blade 48 is exposed. Further movement of the cam 64 is restricted to the circumferential direction or other desired tissue cutting path.

It should be noted that in all the described embodiments springs or resilient elements may be employed to oppose the movement imparted by the inflatable balloons and/or to bias element(s) moved in a first direction by inflation of a balloon actuator so that the element(s) may be moved in the opposite direction as the balloon is allowed to deflate. In one embodiment, a balloon of such a balloon actuator may be coupled to a moving surface, so that deflating the balloon causes the element actuated by inflation of the balloon in a direction opposite to the inflation direction. Known methods of controlling and limiting the movement of the FTRD's movable portions, such as guides and stopping surfaces, may also be included as required in the device.

It will be apparent to those skilled in the art that various modifications and variations can be made in the structure and the methodology of the present invention, without departing from the spirit or scope of the invention. Thus, it is intended that the present invention cover the modifications and variations of this invention provided they come within the scope of the appended claims and their equivalents.

What is claimed is:

1. An actuator for a resection device comprising:
a housing which, when in an operative position, is located within a body lumen of a patient;
an inflatable element having a collapsed configuration and an expanded configuration, wherein a characteristic length of the inflatable element is greater when in the expanded configuration than when in the collapsed configuration, a first surface of the inflatable element contacting the housing and a second surface of the inflatable element contacting a moveable component of the device movably coupled to the housing such that changing the configuration of the inflatable element between the expanded and collapsed configurations moves the moveable component relative to the housing; and
a fluid supply line extending from a distal end connected to the inflatable element to a proximal end which, when the housing is in the operative position, remains outside the body of the patient.

2. The actuator according to claim 1, wherein the moveable component is coupled to an anvil operating in conjunction with a stapling mechanism in the housing, movement of the moveable component relative to the housing moving the anvil relative to the stapling mechanism.

3. The actuator according to claim 1, wherein the movable component is coupled to a staple actuator of a stapling mechanism in the housing, moving the staple actuator relative to the housing firing staples from the stapling mechanism.

4. The actuator according to claim 1, wherein the moveable component is coupled to a telescoping member, movement of the moveable component altering a length of the telescoping member.

5. The actuator according to claim 2, wherein, when the inflatable element is changed from the collapsed to the expanded configuration, the anvil is moved toward the stapling mechanism.

6. The actuator according to claim 5, further comprising a resilient element biasing the anvil away from the stapling mechanism.

7. The actuator according to claim 1, further comprising a hand operated pump coupled to the proximal end of the fluid supply line.

8. The actuator according to claim 1, wherein the fluid supply line extends along an elongated catheter.

9. The actuator according to claim 3, wherein the stapling actuator further comprises a stapling cam having a first end contacting the second surface of the inflatable element and a second end contacting a staple received in the stapling mechanism.

10. The actuator according to claim 1, wherein the movable component is a cutting blade actuator.

11. The actuator according to claim 1, wherein the resectioning device is a full-thickness resectioning device.

12. A resectioning device comprising:
a housing which, when in an operative position, is located within a body lumen of a patient, the housing containing a staple firing mechanism and a tissue cutting blade;
an anvil movably coupled to the housing;
an inflatable element having a collapsed configuration and an expanded configuration, wherein a characteristic length of the inflatable element is greater when in the expanded configuration than when in the collapsed configuration, a first surface of the inflatable element contacting the housing and a second surface of the inflatable element contacting a moveable component of the device movably coupled to the housing such that changing the configuration of the inflatable element between the expanded and collapsed configurations moves the moveable component relative to the housing; and
an inflation fluid lumen extending between a distal end connected to the inflatable element and a proximal end which, when the housing is in the operative position, remains outside the body of the patient.

13. The resectioning device according to claim 12, wherein the moveable component is coupled to the staple firing mechanism so that, when the inflatable element is moved from the collapsed configuration to the expanded configuration, the staple firing mechanism is actuated.

14. The resectioning device according to claim 12, wherein the moveable component is coupled to the tissue cutting blade so that, when the inflatable element is moved from the collapsed configuration to the expanded configuration, the tissue cutting blade is actuated.

15. The resectioning device according to claim 12, wherein the moveable component is coupled to the anvil so that, when the the inflatable element is moved between the collapsed and expanded configurations, the anvil is moved relative to the housing.

16. The resectioning device according to claim 15, further comprising a resilient member biasing the anvil toward an open position in which a first surface of the anvil is separated from the housing.

17. The resectioning device according to claim 12, further comprising a hand operated pump coupled to the proximal end of the inflation fluid supply line.

18. The resectioning device according to claim 12, wherein the resectioning device is a full-thickness resectioning device.

19. An actuator for a resection device comprising:
an inflatable member having a collapsed configuration and an expanded configuration, the inflatable member having a greater characteristic length in the expanded configuration than in the collapsed configuration;
a first surface of the inflatable member contacting a first component fixed to a housing of the resection device;
a second surface of the inflatable member contacting a second component movably coupled to the housing, such that the second component is moved when the inflatable member changes between the collapsed and expanded configurations; and
a fluid supply line connected to the inflatable member for supplying inflation fluid thereto.

20. The actuator according to claim 19, further comprising a resilient element biasing the second component in a direction opposite to the direction in which the second component will be moved by inflation of the inflatable member from the collapsed to the expanded configuration.

21. The actuator according to claim 20, wherein the second component is an anvil of a stapling mechanism of the resection device and wherein the resilient element biases the anvil away from a staple firing mechanism of the stapling mechanism.

22. A resection device comprising:
a housing for insertion into a body lumen, the housing containing a staple firing mechanism and a cutting blade actuation mechanism;
an anvil portion movable relative to the housing between a tissue receiving position and a closed position; and
an expandable component disposed in the housing, the expandable component being moveable between a collapsed configuration and an expanded configuration, a characteristic length of the expandable component being greater in the expanded configuration than in the collapsed configuration.

23. The resectioning device according to claim 22, wherein the expandable component, when moved into the expanded configuration, actuates the staple firing mechanism.

24. The resectioning device according to claim 22, wherein the expandable component, when moved into the expanded configuration, actuates the cutting blade mechanism.

26. The resectioning device according to claim 22, wherein moving the expandable component between the collapsed and the expanded configurations moves the anvil portion relative to a housing of the resection device.

27. The resectioning device according to claim 26, further comprising a resilient member biasing the anvil in a direction opposite to a direction in which the anvil is moved when the expandable component is moved from the collapsed configuration to the expanded configuration.
